(19) United States
(12) Patent Application Publication
(10) Pub. No.: US 2004/0038254 A1
(43) Pub. Date: Feb. 26, 2004

(54) Compositions and methods for detecting nucleic acid methylation

(76) Inventors: Risa Peoples, Palo Alto, CA (US); Reuel van Atta, Mountain View, CA (US)

Correspondence Address: Todd A. Lorenz, DORSEY & WHITNEY LLP, Suite 3400, Four Embarcadero Center, San Francisco, CA, 94111-4187, US

(21) Appl. No.: 10/386,213

(22) Filed: Mar. 10, 2003

Related U.S. Application Data
(60) Provisional application No. 60/362,772, filed on Mar. 8, 2002.

(51) Int. Cl. C12Q 1/68

(52) U.S. Cl. 435/6

(57) ABSTRACT

Methods and compositions are provided for detecting the presence or absence of methylation at methylation sites in a target nucleic acid sequence, utilizing probe sets complementary to first and second binding domains located upstream and downstream of one or more methylation sites of interest in a nucleic acid sequence. Methylation determination may be combined with the detection of additional polymorphisms, such as single nucleotide polymorphisms and/or gene dosage determinations, to provide a more complete genetic profile at a locus or loci of interest.

### FIELD OF THE INVENTION

The present invention relates generally to DNA methylation analysis, and more specifically to detecting the presence of a methyl group at one or more cytosine or adenosine residues in a target sequence, either alone or in combination with other polymorphisms of interest.

### BACKGROUND OF THE INVENTION

Methylation of cytosine is the only known endogenous modification of DNA in eukaryotes, and occurs by the enzymatic addition of a methyl or hydroxymethly group to the carbon-4 or carbon-5 position of cytosine. Costello and Plass, J. Med. Genet. 2001; 38:285-303. In prokaryotes, the nitrogen-6 position of adenosine may also be variably methylated. The DNA methylation pattern is generally established early in life, and has profound epigenetic effects (alteration in gene expression without a change in nucleotide sequence) on the mammalian genome, including transcriptional silencing, genomic imprinting, X chromosome inactivation, and the suppression of parasitic DNA sequences. Robertson and Jones, Carcinogenesis 2000; 21:461-67. Defects or disruptions in the mammalian DNA methylation pattern can lead to disorders such as mental retardation, immune deficiency and sporadic or inherited cancers.

In higher order eukaryotes, the majority of DNA methylation occurs at cytosines located 5′ to guanosine in the CpG dinucleotide, with non-CpG sequences such as 5′-CpNpG-3′ or non-symmetrical 5′-CpA-3′ and 5′-CpT-3′ also exhibiting methylation but at a much lower frequency. Costello, supra. CpGs are not uniformly distributed, and areas of high CpG dinucleotide density, termed “CpG islands,” occur throughout the genome. These CpG islands typically map to gene promoter regions and/or exons, with approximately 50-60% of all genes containing such an island. With the noted exceptions of imprinted genes and several genes on the inactive X chromosome in females, CpGs within CpG islands are normally unmethylated while most CpGs outside CpG islands are methylated. It has been suggested that these patterns of methylation may serve to compartmentalize the genome into transcriptionally active and inactive zones. Id.

The patterns of DNA methylation are thought to reflect two types of gene 5′ regulatory regions in the genome. Singal and Ginder, Blood 1999; 93:4059-70. While about 60% of the genes having CpG islands represent mainly housekeeping genes with a broad tissue pattern of expression, approximately 40% exhibit a tissue-specific pattern of expression. Promoter region CpG islands are usually unmethylated in all normal tissues, regardless of the transcriptional activity of the gene, with the exception of non-transcribed genes on the inactive X chromosome and imprinted autosomal genes where one of the parental alleles may be methylated. Tissue specific genes without CpG islands are variably methylated, often in a tissue-specific pattern, and methylation is usually inversely correlated with the transcriptional status of the gene. Id.

In view of the many epigenetic effects involved in DNA methylation, there is a rapidly growing interest in studying variations in methylation patterns. Methylation analysis has proven useful in studying human diseases associated with imprinted regions and defects in imprinted genes or their epigenetic regulation, such as Beckwith-Wiedemann syndrome (BWS) on human chromosome 11p15 and the Prader-Willi and Angelman syndromes (PWS/AS) on chromosome 15q11-q13. The study of methylation is also particularly pertinent to cancer research as molecular alterations during malignancy may result from a local hypermethylation of tumor suppressor genes, along with a genome wide demethylation. Schulze et al., Nat. Genet. 1996; 12:452-454. Unfortunately, however, current methodologies employed in DNA methylation analysis are insufficient in many respects.

Early techniques utilized to study site-specific DNA methylation combined Southern hybridization with methylation-sensitive type II restriction enzymes, relying on the inability of the enzymes to cleave sequences containing one or more methylated CpG sites. Epstein et al., Nature 1978; 274:500-503. While these methods do provide an assessment of the overall methylation status of CpG islands, including some quantitative analysis, they require large amounts of high molecular weight DNA (generally 5 μg or more), can detect methylation only if present in greater than a few percent of the alleles and can only provide information about those CpG sites found within sequences recognized by methylation-sensitive restriction enzymes. Singal, supra.

More recent methods for studying site-specific DNA methylation generally rely on a methylation-dependent modification of the original genomic DNA prior to an amplification step. Singer-Sam et al. sought to improve sensitivity by combining the use of methylation-sensitive restriction enzymes with the polymerase chain reaction (PCR). Singer-Sam et al., Mol. Cell. Biol. 1990; 10:4987. This method, however, like the Southern-based approach discussed above, can only monitor CpG methylation in methylation-sensitive restriction sites. Moreover, the method is not quantitative and is very prone to error, since any uncleaved DNA will be amplified by PCR yielding a false positive result for methylation. Singal, supra.

Frommer et al. introduced a procedure based on bisulfite-induced oxidative deamination of genomic DNA, which changes unmethylated cytosines to uracil while leaving methylated cytosines alone. Frommer et al., Proc. Nat'l Acad. Sci. USA 1992; 89:1827. This altered DNA can then be amplified and sequenced, providing detailed information within the amplified region of the methylation status of all CpG sites. Unfortunately, however, the method is technically rather difficult and labor-intensive, and, without cloning of the amplified products, is less sensitive than the original Southern analysis. Herman et al., Proc. Nat'l Acad. Sci. USA 1996; 93:9821-26.

A number of related methods have been subsequently developed to more rapidly detect 5-mC based on the bisulfite deamination reaction in combination with PCR amplification. The general utility of these methods is limited, however, in that they are suitable for studying only limited numbers of CpG dinucleotides that are either found within or immediately adjacent to the PCR primer sequences (e.g., methylation-specific PCR (MSP) described in Herman et al., supra; and methylation-sensitive single nucleotide primer extension (Ms-SNuPE) described in Gonzalgo and Jones, Nuc. Acids Res. 1997; 25:2529-31) or within a restriction enzyme recognition sequence (Xiong and Laird, Nuc. Acids Res. 1997; 25:2532). See Singal, supra. The MSP technique of Herman et al. has been subsequently described and patented in U.S. Pat. Nos. 6,265,171; 6,017,704 & 5,786,146, the disclosures of which are expressly incorporated by reference herein in their entirety.

Thus, there is a substantial need in the art for an improved method for determining the methylation status of a known or suspected methylation site in a site-specific manner. Preferably, the method will enable the rapid and reliable detection of 5-mC at one or more methylation sites of interest in a gene, either alone or in combination with the detection of other polymorphic sequences of interest. Thus, the method should be capable of identifying candidate disease genes by concurrently detecting altered methylation patterns along with additional polymorphisms in the same platform.

Relevant Literature

The application of the methylation-sensitive restriction enzyme Southern blotting technique to the PWS/AS locus is described in Dittrich et al., Hum. Genet. 1992; 90:313-315; Driscoll et al., Genomics 1992; 13:917-924; and Glenn et al., Hum. Mol. Genet. 1993; 2:2001-2005. Singer-Sam et al., Nucl. Acids. Res. 1990; 18:687 discloses digestion with methylation sensitive enzymes followed by PCR, while Chotai et al., J. Med. Genet. 1998; 35:472-475 describe the application of this technique to the PWS/AS locus. Bisulfite modification of a genomic DNA template (MSPCR) using allele-specific primers is disclosed in Herman et al., Proc. Natl. Acad. Sci. USA 1996; 93:9821-9826; Kubota et al., Nat. Genet. 1997; 16:16-17; and Zeschnigk et al., Eur. J. Hum. Genet. 1997; 5:94-98. MSPCR using common primers followed by restriction digestion of amplicons is described by Velinov et al., Mol. Genet. Metab. 2000; 69:81-83. All references referred to herein are expressly incorporated by reference.

### SUMMARY OF THE INVENTION

In accordance with the objects outlined above, the present invention provides a method for determining the methylation status of a target nucleic acid sequence in a sample, wherein said target nucleic acid sequence comprises a first and a second binding domain and at least one methylation site. In one embodiment, the method comprises the steps of: a) adding a methylation-related digestion enzyme to said sample; b) adding a capture probe having a sequence substantially complementary to at least a portion of said first binding domain and a reporter probe having a sequence substantially complementary to at least a portion of said second binding domain, wherein said first and second binding domains are separated by said methylation site in said target sequence; c) capturing said capture probe; and d) detecting said reporter probe to determine methylation status at said methylation site.

In a preferred embodiment, the methylation-related enzyme comprises a methylation-sensitive enzyme, and detection of the reporter probe indicates methylation at the methylation site. In an alternative embodiment, the methylation-related enzyme comprises a methylation-dependent enzyme, and detection of the reporter probe indicates a lack of methylation at the methylation site.

In another preferred embodiment, the capture and reporter probes comprise first and second detectable labels respectively. In one embodiment, the first detectable label comprises a capture molecule. In a further embodiment, the second detectable label comprises a reporter molecule.

In one aspect, the capture and reporter probes are crosslinkable probes comprising at least one crosslinking agent. In this embodiment, the crosslinkable probes are activated to crosslink to their respective binding domains prior to capture of the capture probe and a high-stringency wash step may be employed. In a preferred aspect, the crosslinkable probes comprise a photo-activatible crosslinking agent.

In one embodiment, a method for genotyping a target sequence in a sample is provided, wherein said target sequence comprises a dosage region and a methylation site flanked by first and second binding domains, the method comprising the steps of: a) adding a methylation-related digestion enzyme to said sample; b) hybridizing said first and second binding domains to a first probe mixture to form at least one first hybridization complex, said first probe mixture comprising at least one methylation capture probe having a sequence substantially complementary to at least a portion of said first binding domain and at least one methylation reporter probe having a sequence substantially complementary to at least a portion of said second binding domain, wherein said first and second binding domains are separated by said methylation site in said target sequence; c) hybridizing said dosage region to a second probe mixture to form at least one second hybridization complex, said second probe mixture comprising at least one dosage reporter probe comprising a detectable label capable of producing a dosage signal and a sequence substantially complementary to at least a portion of said dosage region; d) capturing said at least one methylation capture probe, and e) determining the copy number of said dosage region based on the ratio of said dosage region to a diploid signal and detecting said methylation reporter probe to determine the methylation status of the target.

In a further embodiment, the method comprises the additional steps of hybridizing a third probe mixture to a diploid region in said sample and performing said detecting step to obtain said diploid signal; wherein said third probe mixture comprises at least one diploid reporter probe having a sequence complementary to at least a portion of said diploid region and a detectable label capable of producing said diploid signal.

In a preferred embodiment, the methylation-related enzyme comprises a methylation-sensitive enzyme, and detection of the reporter probe indicates methylation at the methylation site. In an alternative embodiment, the methylation-related enzyme comprises a methylation-dependent enzyme, and detection of the reporter probe indicates a lack of methylation at the methylation site.

In another preferred embodiment, the capture and reporter probes comprise first and second detectable labels respectively. In one embodiment, the first detectable label comprises a capture molecule. In a further embodiment, the second detectable label comprises a reporter molecule.

In one aspect, the capture and reporter probes are crosslinkable probes comprising at least one crosslinking agent. In a further aspect, the crosslinkable probes are activated to crosslink to their respective binding domains prior to capture of the capture probe, whereby said first hybridization complex becomes covalently crosslinked when said first and second binding sites are present in said sample, and said second hybridization complex becomes covalently crosslinked when said dosage region is present in said sample. In a preferred aspect, the crosslinkable probes comprise a photo-activatible crosslinking agent.

In a further embodiment the instant invention provides a method for genotyping a target sequence in a sample, wherein the target sequence comprises a dosage region and a methylation site flanked by first and second binding domains, the method comprising: a) adding a methylation-related digestion enzyme to the sample; b) hybridizing the first and second binding domains to a first crosslinkable probe mixture to form at least one first hybridization complex, the first crosslinkable probe mixture comprising at least one methylation capture probe having a sequence substantially complementary to at least a portion of the first binding domain and at least one methylation reporter probe having a sequence substantially complementary to at least a portion of the second binding domain, wherein said first and second binding domains are separated by the methylation site in said target sequence; c) hybridizing the dosage region to a second crosslinkable probe mixture to form at least one second hybridization complex, the second crosslinkable probe mixture comprising at least one dosage reporter probe comprising a crosslinking agent, a detectable label capable of producing a dosage signal and a sequence substantially complementary to at least a portion of the dosage region; d) activating the crosslinking agent, whereby the first hybridization complex becomes covalently crosslinked when the first and second binding domains are present in said sample, and the second hybridization complex becomes covalently crosslinked when the dosage region is present in the sample; e) washing the crosslinked first and second hybridization complexes at least once under high-stringency conditions; and f) detecting the dosage signal to determine the copy number of the dosage region and detecting the methylation reporter probe to determine the methylation status of the target.

In another embodiment, the instant invention provides a method for genotyping a target sequence in a sample, wherein the target sequence comprises a methylation site flanked by a first and a second binding domain and an interrogation region comprising an interrogation position, the method comprising: a) adding a methylation-related digestion enzyme to the sample; b) hybridizing the first and second binding domains to a first crosslinkable probe mixture to form at least one first hybridization complex, the first crosslinkable probe mixture comprising at least one methylation capture probe having a sequence substantially complementary to at least a portion of the first binding domain and at least one methylation reporter probe having a sequence substantially complementary to at least a portion of the second binding domain, wherein the first and second binding domains are separated by the methylation site in the target sequence; c) hybridizing the interrogation region to a second crosslinkable probe mixture to form at least one second hybridization complex, the second crosslinkable probe mixture comprising at least one allele-specific detection probe comprising a crosslinking agent, a detectable label capable of producing an interrogation signal and a sequence substantially complementary to the sequence upstream and downstream of the interrogation position in the interrogation region; d) activating crosslinking agent, whereby first hybridization complex becomes covalently crosslinked when the first and second binding domains are present in the sample, and the second hybridization complex becomes covalently crosslinked when the detection position is perfectly complementary to the interrogation position; e) washing the crosslinked first and second hybridization complexes at least once under high-stringency conditions; and f) detecting the methylation reporter probe to determine the methylation status of the target and detecting the interrogation signal to determine the identity of the interrogation position.

### BRIEF DESCRIPTION OF THE DRAWINGS

FIG. 1 is a diagram illustrating a crossover event that can occur during meiosis and lead to abnormal gene copy number.

FIG. 2 is a diagram illustrating transcription versus silencing of the SNRPn gene on chromosome 15, which is implicated in the Prader-Willi syndrome.

FIG. 3 is a diagram illustrating the design of capture and reporter probe sets directed to determining methylation status and gene dosage at 15q11-13, as well as for the diploid control locus at 4q25.

FIG. 4 is a diagram illustrating the design of alternative capture and reporter probes for determination of methylation status and dosage at 15q11-13, without crosslinkers.

FIG. 5 is a diagram illustrating the design of capture and reporter probe sets directed to determining methylation status of the tumor suppressor gene p53 sequence.

### DETAILED DESCRIPTION OF THE PREFERRED EMBODIMENTS

The present invention provides methods and compositions for detecting the presence or absence of nucleic acid methylation in a target sequence, either alone or in combination with the detection of other polymorphisms of interest. The method involves determining the methylation status of one or more methylation sites in a sample, utilizing nucleic acid probes in conjunction with methylation-sensitive restriction enzymes. As described in more detail herein, the presence or absence of methylation will be detected based on the separation of the capture and reporter probes of the present invention and the consequent loss of signal.

As used herein, “methylation status” refers to the presence or absence of a methyl or hydroxymethyl group attached to the carbon-4 position (4-mC) or carbon-5 position (5-mC) of cytosine in eukaryotes. Methylation of cytosine and/or the nitrogen-6 position of adenosine (6-mA) in prokaryotes is also contemplated by the present invention. By “methylation site” is meant a nucleic acid sequence in which methylase may optionally add a methyl group to an adenine or cytosine residue. The methylation site generally further comprises a methylation-related restriction enzyme binding site. Such enzymes can be either methylation-sensitive or methylation-dependent in their function. In preferred embodiments a methylation-sensitive digestion enzyme is used, in which case the presence of a methyl group at a methylation-sensitive restriction enzyme binding site will typically render the site resistant to restriction by a methylation-sensitive digestion enzyme. A much smaller complement of methylation-dependent restriction endonucleases that preferentially cleave methylated sequences have also been described (McClelland et al., Nucl. Acids Res. 1995; 22:3640-59), and are also contemplated for use in the present invention. A typical example is DpnI, which cleaves 6-methyl adenosine residues when found on the consensus sequence GATC. While the ensuing discussion generally refers to the use of methylation-sensitive restriction endonucleases, it is understood that a methylation-dependent restriction endonuclease can also be used to provide equal methylation versus unmethylation discrimination.

In one embodiment, the invention provides a method for determining the methylation status of one or more known or suspected methylation sites in a sample for one or more genes of interest. Generally, the method comprises combining a probe mixture comprising a first set of capture and reporter probes with a sample comprising a target sequence, which may be present as a major component of the DNA from the target or as one member of a complex mixture. A target sequence having a methylation region comprising one or more methylation site(s) of interest is initially provided in double-stranded form, and may further comprise a dosage region, a control region and/or an interrogation region as described herein. The first set of methylation capture and reporter probes are characterized by having known sequences derived from the gene or genes of interest, with complementarity to first and second binding domains in the methylation region, as explained below. In a further embodiment, additional probe sets directed to other polymorphic sequences and diploid control locus are also provided.

In a preferred embodiment, the capture and reporter probes further comprise first and second detectable labels, respectively. The first detectable label of the capture probe preferably comprises a molecule that can be captured on a solid support, e.g., biotin, whereas the second detectable label of the reporter probe preferably comprises a reporter molecule, e.g., a fluorophore, an antigen, or other binding-pair partner useful for direct or indirect detection methods. In a particularly preferred embodiment, the first detectable label allows for separation of the capture probe-target complexes, such as, e.g., a biotinylated probe exposed to streptavidin-coated beads, whereas the second detectable label provides for quantification of signal strength, such as, e.g., fluorescein. The capture probe is then captured and the reporter probe is detected to determine methylation.

As described herein, if the enzyme has cut the sample, the reporter probe will be disassociated from the capture probe, and no signal will be detected. In the preferred embodiments employing methylation-sensitive digestion enzymes, detection of the reporter probe correlates with methylation at the methylation site. In alternative embodiments utilizing employing methylation-dependent digestion enzymes, detection of the reporter probe correlates with a lack of methylation at the methylation site.

Following the methods of the present invention, one may also determine methylation status in parallel with the detection of one or more additional types of polymorphism that may be present in a gene or genes of interest. The polymorphism may be either inherited or spontaneous, germline or somatic, or a marker of interspecies variation. Polymorphisms or mutations of interest include those related to gene dosage abnormalities such as deletions and duplications, as well as substitutions, insertions, translocations, rearrangements, variable number of tandem repeats, short tandem repeats, retrotransposons such as Alu and long interspersed nucleotide element (LINE), single-nucleotide polymorphisms (SNPs) and the like. By convention, sequence variants present at frequencies less than 1% are generally considered mutations, whereas those present at higher frequencies are considered polymorphisms. As used herein, the term “polymorphism” means any DNA sequence variation of any type or frequency.

In a preferred embodiment, the additional polymorphism detected following the methods of the present invention relates to gene dosage. As used herein, gene dosage refers to the quantitative determination of gene copy number present in an individual's genome. Because the normal human genome is diploid, the normal gene dosage for non X-linked genes is two. Whole gene and larger (microscopic and submicroscopic subchromosomal) deletions and duplications (gene dosage of one and three or more, respectively) confer specific phenotypes, and their diagnosis can be of critical clinical importance. As described herein, the present invention also provides methods and compositions for rapidly and accurately determining the gene copy number of genomic regions subject to these types of duplication and/or deletion events, referred to generally herein as “dosage regions.” Preferably, in this embodiment the sample further comprises a diploid control locus, termed a “diploid region,” and the gene copy number is determined from the ratio of a dosage signal generated by a probe set directed to the dosage region and a diploid signal generated by a probe set directed to the diploid region, as described further herein. Additional probe sets directed to other polymorphisms or mutations in the gene or genes of interest may also be employed concurrently in the same platform for the same clinical sample, providing a complete genetic profile of a given locus in parallel with the determination of methylation status.

As will be appreciated by those in the art, the sample may comprise any number of things, including, but not limited to, bodily fluids (including, but not limited to, blood, urine, serum, lymph, saliva, anal and vaginal secretions, perspiration, and semen, of virtually any organism, with mammalian samples being preferred and human samples being particularly preferred); research samples; purified samples, such as purified genomic DNA, RNA, etc.; raw samples, such as bacteria, virus, genomic DNA, mRNA, etc. The sample may comprise individual cells, including primary cells (including bacteria), and cell lines, including, but not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, 923, HeLa, WI-38, Weri-1, MG-63, etc. See the ATCC cell line catalog, hereby expressly incorporated by reference. As will be appreciated by those in the art, virtually any experimental manipulation may have been done on the sample.

By “nucleic acid” or “oligonucleotide” or grammatical equivalents herein means at least two nucleotides covalently linked together. As will be appreciated by those skilled in the art, various modifications of the sugar-phosphate backbone may be done to facilitate the addition of labels, or to increase the stability and half-life of such molecules in physiological environments. The nucleic acids may be single-stranded or double-stranded, as specified, or contain portions of both double-stranded or single-stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleotide” includes nucleotides as well as nucleoside and nucleotide analogs, and modified nucleosides such as labeled nucleosides. In addition, “nucleotide” includes non-naturally occurring analog structures. Thus, for example, the individual units of a peptide nucleic acid (PNA), each containing a base, are referred to herein as a nucleotide. The term “nucleotide” also encompasses locked nucleic acids (LNA). BVraasch and Corey, Chem. Biol. 2001; 8(1): 1-7. Similarly, the term “nucleotide” (sometimes abbreviated herein as “NTP”), includes both ribonucleic acid and deoxyribonucleic acid (sometimes abbreviated herein as “dNTP”).

The compositions and methods of the invention are directed to determining the methylation status, dosage and/or genotype of target sequences. The terms “target sequence” or “target nucleic acid” or grammatical equivalents herein mean a nucleic acid sequence. In a preferred embodiment, the target sequence comprises a methylation region, generally having at least one methylation site of interest. In another embodiment, the target sequence further comprises an additional polymorphism of interest, e.g., a deletion or duplication (termed a “dosage region”) or a SNP. Alternatively, the sample may comprise a plurality of distinct target sequences, each having one or more polymorphisms of interest. By “plurality” as used herein is meant at least two.

The target nucleic acid may come from any source, either prokaryotic or eukaryotic, usually eukaryotic. The source may be the genome of the host, plasmid DNA, viral DNA, where the virus may be naturally occurring or serving as a vector for DNA from a different source, a PCR amplification product, or the like. The target DNA may be a particular allele of a mammalian host, an MHC allele, a sequence coding for an enzyme isoform, a particular gene or strain of a unicellular organism, or the like. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a genotyping or amplification reaction such as a ligated circularized probe, an amplicon from an amplification reaction such as PCR, etc. Thus, for example, a target sequence from a sample is amplified to produce a secondary target (amplicon) that is detected. Alternatively, what may be amplified is the probe sequence, although this is not generally preferred. Thus, as will be appreciated by those in the art, the complementary target sequence may take many forms. For example, it may be contained within a larger nucleic acid sequence, i.e. all or part of a gene or mRNA, a restriction fragment of a cloning vector or genomic DNA, among others. As is outlined more fully below, probes are made to hybridize to target and/or control sequences to determine the presence, sequence, quantity or methylation status of a target sequence in a sample. Generally speaking, the term “target sequence” will be understood by those skilled in the art.

If required, the target sequence is prepared using known techniques. For example, the sample may be treated to lyse the cells, using known lysis buffers, sonication, electroporation, etc., with purification and amplification occurring as needed, as will be appreciated by those in the art. The sample may be a cellular lysate, isolated episomal element, e.g., YAC, plasmid, etc., virus, purified chromosomal fragments, cDNA generated by reverse transcriptase, amplification product, mRNA, etc. Depending upon the source, the nucleic acid may be freed of cellular debris, proteins, DNA (if RNA is of interest), RNA (if DNA is of interest), size selected, gel electrophoresed, restriction enzyme digested, sheared, fragmented by alkaline hydrolysis, or the like. Importantly, however, and unlike the prior art, the benefits of improved sensitivity and reproducibility may be obtained following the methods of the present invention even without such additional DNA purification steps.

The target sequence may be of any length, with the understanding that longer sequences are more specific. In one embodiment, the target nucleic acid is provided with an average size in the range of about 0.25 to 3 kb. Nucleic acids of the desired length can be achieved, particularly with DNA, by restriction enzyme digestion, use of PCR and primers, boiling of high molecular weight DNA for a prescribed time, and the like. Desirably, at least about 80 mol %, usually at least about 90 mol % of the target sequence, will have the same size. For restriction enzyme digestion, a frequently cutting enzyme may be employed, usually an enzyme with a four-base recognition sequence, or combination of restriction enzymes may be employed, where the DNA will be subject to complete digestion.

In the preferred embodiment of the methods of the present invention directed to determining methylation status, the method specifically includes a digestion step utilizing a “methylation-related digestion enzyme,” by which is meant an enzyme that has sequence specificity in addition to methylation sensitivity. Thus, “methylation-related” is defined herein to include both methylation-sensitive and methylation-dependent restriction endonucleases. In the case of methylation-dependent enzymes, the enzyme will preferentially cut in the presence of methylated sequences, as described in McClelland et al., supra. In preferred embodiments, methylation-sensitive enzymes are utilized which will not cut if the sequence is methylated, and will cut if the sequence is non-methylated. In a particularly preferred embodiment the methylation-sensitive enzyme comprises Hpa II, which recognizes 5′-CCGG-3′. The digestion is blocked by methylation at either C. Additional exemplary methylation-sensitive digestion enzymes suitable for use in the present invention are included in Table 1 below:

TABLE 1

| Enzyme | Sequence |
|---|---|
| BclI | TGaTCA |
| BspPI | GGaTC |
| MboI | GaTC |
| Bsu15I | 5′ . . . ATCGaT C . . . 3′ |
| | 3′ . . . TAGCTa G . . . 5′ |
| Hin4I | 5′ . . . GaTC(N)₄VTC . . . 3′ |
| | 3′ . . . CTaG(N)₄BAG . . . 5′ |
| | 5′ . . . GAY(N)₄GaTC . . . 3′ |
| | 3′ . . . CTR(N)₄CTaG . . . 5′ |
| HphI | 5′ . . . GGTGa TC . . . 3′ |
| | 3′ . . . CCACT aG . . . 5′ |
| MboII | 5′ . . . GAAGa TC . . . 3′ |
| | 3′ . . . CTTCT aG . . . 5′ |
| TaqI | 5′ . . . TCGa TC . . . 3′ |
| | 3′ . . . AGCT aG . . . 5′ |
| XbaI | 5′ . . . TCTAGa TC . . . 3′ |
| | 3′ . . . AGATCT aG . . . 5′ |
| Acc65I | 5′ . . . CcWGG TA CcWGG . . . 3′ |
| | 3′ . . . GGWcC AT GGWcC . . . 5′ |
| Bme1390I | 5′ . . . Cc AGG . . . 3′ |
| | 3′ . . . GG TcC . . . 5′ |
| BseLI | 5′ . . . Cc WGGNCcW GG . . . 3′ |
| | 3′ . . . GG WcCNGGW cC . . . 5′ |
| Bsp120I | 5′ . . . GGGCCc WGG . . . 3′ |
| | 3′ . . . CCCGGG WcC . . . 5′ |
| BspLI | 5′ . . . CcWGG NN CcWGG . . . 3′ |
| | 3′ . . . GGWcC NN GGWcC . . . 5′ |
| CaiI | 5′ . . . CAGNNCcTGG . . . 3′ |
| | 3′ . . . GTCNNGGTcC . . . 5′ |
| CfrI | 5′ . . . YGGCc WGG . . . 3′ |
| | 3′ . . . RCCGG WcC . . . 5′ |
| Cfr13I | 5′ . . . GGNCc WGG . . . 3′ |
| | 3′ . . . CCNGG WcC . . . 5′ |
| Eco47I | 5′ . . . GGWCc WGG . . . 3′ |
| | 3′ . . . CCWGG WcC . . . 5′ |
| Eco57MI | 5′ . . . CcTGG AG . . . 3′ |
| | 3′ . . . GGAcC TC . . . 5′ |
| Eco147I | 5′ . . . AGGCcT GG . . . 3′ |
| | 3′ . . . TCCGGA cC . . . 5′ |
| EcoO109I | 5′ . . . RGGNCcT GG . . . 3′ |
| | 3′ . . . YCCNGGA cC . . . 5′ |
| GsuI | 5′ . . . CTCcAG G . . . 3′ |
| | 3′ . . . GAGGTc C . . . 5′ |
| MIsI | 5′ . . . TGGCcA GG . . . 3′ |
| | 3′ . . . ACCGGT cC . . . 5′ |
| Psp5II | 5′ . . . RGGWCcT GG . . . 3′ |
| | 3′ . . . YCCWGGA cC . . . 5′ |
| Van91I | 5′ . . . CcAGGNNNTGG . . . 3′ |
| | 3′ . . . GGTcCNNNACC . . . 5′ |

Codes:

a - N⁶-methyladenine (m⁶A)
c - 5-methylcytosine (m⁵C)
R = G or A; H = A, C or T; Y = C or T; V = A, C or G; W = A or T; B =
C, G or T;
M = A or C; D = A, G or T; K = G or T; N = G, A, T or C; S = C or G.

Preferably, after the digestion step the double-stranded target nucleic acids are then denatured to render them single-stranded, so as to permit hybridization of the capture and reporter probes of the invention. A preferred embodiment utilizes a thermal step, generally by raising the temperature of the reaction to about 95° C. in an alkaline environment, although chemical denaturation techniques may also be used. Where chemical denaturation has occurred, normally the medium will then be neutralized to permit hybridization. Various media can be employed for neutralization, particularly using mild acids and buffers, such as acetic acid, citric acid, etc. The particular neutralization buffer employed is selected to provide the desired stringency for hybridization to occur during the subsequent incubation.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents that may be included in the assays. These reagents include salts, buffers, neutral proteins, e.g., albumin, detergents, etc., that may be used to facilitate optimal hybridization and detection, and/or reduce non-specific interactions. Also reagents that otherwise improve the efficacy of the assay, such as protease inhibitors, nuclease inhibitors, anti-microbial agents, etc., may be used, depending on the sample preparation methods and purity of the target.

In a preferred embodiment, a method for determining the methylation status of a methylation site in a target sequence is described, wherein the target sequence comprises a region having one or more methylation sites to be analyzed, generally referred to herein as the “methylation region.” Preferably, the target sequence will comprise not more than 2 kb, with the methylation site anywhere from 1-300 base pairs from either side. More preferably, the methylation site is anywhere from 1-150 base pairs from either side. Most preferably, the methylation site is anywhere within 100 base pairs from either side.

The method comprises the steps of combining the sample containing the target sequence with a methylation-related enzyme, denaturing and then adding at least one capture probe and at least one reporter probe. The methylation region of the target sequence comprises a first binding domain, which is substantially complementary to the at least one capture probe, and a second binding domain, which is substantially complementary to the at least one reporter probe, along with one or more methylation sites of interest. The first and second binding domain(s) are separated by the methylation site, i.e., if the first binding domain is located 5′ of the methylation site of interest then the second domain is located 3′ of the methylation site, and vice-versa. The capture probe(s) are then captured and the presence of the reporter probe detected in the captured complex. The presence or absence of a signal from the reporter probe(s) will indicate the methylation status of the methylation site, depending on the type of methylation-related enzyme utilized. Probes designed to hybridize with a methylation region in a target sequence are also generally referred to herein as “methylation probes.”

In a further embodiment, the method further comprises determining methylation status in combination with gene dosage, wherein the target sequence further comprises at least a portion of a genomic sequence that is known to be subject to deletion or duplication events, generally referred to herein as the “dosage region.” The dosage region will generally comprise a plurality of nucleotides, and more preferably, a plurality of contiguous nucleotides. As used herein, the corresponding region in the probe sequence that hybridizes with the dosage region or other sequence of interest is termed the “detection region.” Probes designed to hybridize with a dosage region in a target sequence are also generally referred to herein as “dosage probes.”

In a particularly preferred embodiment, the above method further comprises the parallel detection of an additional polymorphism of interest, such as, e.g., a parallel genotyping reaction. As is more fully outlined below, an interrogation region having a position for which sequence information is desired, generally referred to herein as the “interrogation position,” may be detected using additional probe sets complementary to portions of the interrogation region as described herein. In one such embodiment, the interrogation position is a single nucleotide, although in some embodiments, it may comprise a plurality of nucleotides, either contiguous with each other or separated by one or more nucleotides within the interrogation region. As used herein, the corresponding probe base that basepairs with the interrogation position base in a hybridization complex is termed the “detection position.” In the case where the detection position is a single nucleotide, the NTP in the probe that has perfect complementarity to the detection position is called a “detection NTP.” Probes designed to hybridize with at least a portion of the interrogation region in a target sequence are generally referred to herein as “detection probes,” while the subset of such probes comprising a detection position are referred to herein as “allele-specific detection probes.”

“Mismatch” is a relative term and meant to indicate a difference in the identity of a base at a particular position, termed the “interrogation position” herein, between two sequences. In general, sequences that differ from wild type sequences are referred to as mismatches. However, particularly in the case of SNPs, what constitutes “wild type” may be difficult to determine as multiple alleles can be observed relatively frequently in the population, and thus “mismatch” in this context requires the artificial adoption of one sequence as a standard. Thus, for the purposes of this invention, sequences are referred to herein as “perfect match” and “mismatch.” “Mismatches” are also sometimes referred to as “allelic variants.” The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation. The term “allelic variant of a polymorphic region of a gene” refers to a region of a gene having one of several nucleotide sequences among individuals of the same species.

The present invention provides both capture and reporter probes that hybridize to regions of interest within a target sequence or a plurality of target sequences as described herein. In general, probes of the present invention are designed to be complementary to methylation, dosage, diploid and/or interrogation regions of target sequence(s) (either the target sequence of the sample or to other probe sequences), such that hybridization occurs between the target and the probes of the present invention. This complementarity need not be perfect; there may be any number of base-pair mismatches that will interfere with hybridization between the target sequence and the corresponding detection regions in the probes of the present invention. However, if the number of mutations is so great that no hybridization can occur under even the least stringent of hybridization conditions, the sequence is not a complementary target sequence. Thus, by “substantially complementary” herein is meant that the probe sequences are sufficiently complementary to the corresponding region of the target sequence (e.g. methylation, dosage, diploid or interrogation region) to hybridize under the selected reaction conditions.

Hybridization generally depends on the ability of denatured DNA to anneal when complementary strands are present in an environment below their melting temperature. The higher the degree of desired complementarity between the probe sequence and the region of interest, the higher the relative temperature that can be used. As a result, it follows that higher relative temperatures would tend to make the reaction conditions more stringent, whereas lower temperatures less so. For additional details and explanation of stringency of hybridization reactions, see Current Protocols in Molecular Biology, Ausubel et al. (Eds.).

Generally, the length of the probe and its GC content will determine the thermal melting point (Tm) of the hybrid, and thus the hybridization conditions necessary for obtaining specific hybridization of the probe to the region of interest. These factors are well known to a person of skill in the art, and can also be tested experimentally. The Tm is the temperature (under defined ionic strength and pH) at which 50% of the target sequence hybridizes to a probe. An extensive guide to the hybridization of nucleic acids is found in Tijssen, Hybridization with Nucleic Acid Probes: Theory and Nucleic Acid Probes, Vol. 1, 1993. Generally, stringent conditions are selected to be about 5° C. lower than the Tm for the specific sequence at a defined ionic strength and pH. Highly stringent conditions are selected to be greater than or equal to the Tm point for a particular probe.

Sometimes the term “dissociation temperature” (“Td”) is used to define the temperature at which half of the probe is dissociated from a target nucleic acid. In any case, a variety of techniques for estimating the Tm or Td are available, and generally described in Tijssen, supra. Typically, G-C base pairs in a duplex are estimated to contribute about 3° C. to the Tm, whereas A-T base pairs are estimated to contribute about 2° C., up to a theoretical maximum of about 80-100° C. However, more sophisticated models of Tm and Td are available and appropriate in which G-C stacking interactions, solvent effects, and the like are taken into account. For example, probes can be designed to have a desired dissociation temperature by using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5° C. Tibanyenda et al., Eur. J. Biochem. 1984; 139(1):19-27 and Ebel et al., Biochemistry 1992; 31(48):12083-1286. More importantly, it is understood that as the length of the complementary region increases, the effect of a single base mismatch on overall duplex stability decreases. Thus, where there is a likelihood of mismatches between the probe sequence and the target sequence, it may be advisable to include a longer complementary region in the probe. Alternatively, where one is probing a known interrogation position with a plurality of allele-specific detection probes, it may be advisable to include a shorter complementary region in the probes to improve discrimination.

Thus, the specificity and selectivity of the probe can be adjusted by choosing proper lengths for the complementary regions and appropriate hybridization conditions. When the sample is genomic DNA, e.g., mammalian genomic DNA, the selectivity of the probe sequences must be high enough to identify the correct sequence in order to allow processing directly from genomic DNA. However, in situations in which a portion of the genomic DNA is first isolated from the rest of the DNA, e.g., by separating one or more chromosomes from the rest of the chromosomes, the selectivity or specificity of the probe may become less important.

The length of the probe, and therefore the hybridization conditions, will also depend on whether a single probe is hybridized to the target sequence, or several probes. In a preferred embodiment, several probes are used and all the probes are hybridized simultaneously to the target sequence. With this embodiment, it is desirable to design the probe sequences such that their Tm or Td is similar, such that all the probes will hybridize specifically to the target sequence. These conditions can be determined by a person of skill in the art, by taking into consideration the factors discussed above.

A variety of hybridization conditions may be used in the present invention, including high-, moderate- and low-stringency conditions; see, e.g., Sambrook et al., Molecular Cloning. A Laboratory Manual, 2nd ed., 1989, and Short Protocols in Molecular Biology, Ausubel et al (Eds.), 1992, hereby incorporated by reference. Stringent conditions are sequence-dependent, and will differ depending on specific circumstances. Longer sequences hybridize more specifically at higher temperatures. Stringent conditions will be those in which the salt concentration is less than about 1.0 M sodium ion, typically about 0.01 to 1.0 M sodium ion concentration (or other salts) at pH 7.0 to 8.3, and the temperature is at least about 30° C. for short probes (e.g., 10 to 50 nucleotides) and at least about 60° C. for long probes (e.g., greater than 50 nucleotides) in an entirely aqueous hybridization medium. Stringent conditions may also be achieved with the addition of helix destabilizing agents such as formamide. The hybridization conditions may also vary when a non-ionic backbone, e.g., PNA is used, as is known in the art.

Thus, the assays are generally run under stringency conditions that allow formation of the hybridization complex only in the presence of target. Stringency can be controlled by altering a step parameter that is a thermodynamic variable, including, but not limited to, temperature, formamide concentration, salt concentration, chaotrope salt concentration, pH, organic solvent concentration, etc. These parameters may also be used to control non-specific binding, as is generally outlined in U.S. Pat. No. 5,681,697. Thus it may be desirable to perform certain steps at higher stringency conditions to reduce non-specific binding, as described herein. The skilled artisan will recognize how to adjust the temperature, ionic strength, etc. as necessary to accommodate factors such as probe length and the like.

As will be appreciated by those in the art, the capture and reporter probes of the invention can take on a variety of configurations. The desired probe will have a sequence of at least about 10, more usually at least about 15, preferably at least about 16 or 17 and usually not more than about 1 kilobases (kb), more usually not more than about 0.5 kb, preferably in the range of about 18 to 200 nucleotides (nt), and frequently not more than 50 nt, where the probe sequence is substantially complementary to the desired target sequence or control locus.

In a preferred embodiment, particularly suited for detecting nucleic acid methylation, the sequences of a first set of capture and reporter probes are selected so as to be substantially complementary to at least a portion of first and second binding domains, respectively, within a methylation region in a gene or genes of interest. The methylation status of a methylation site located within the methylation region may then be assayed for by detecting the signal from the reporter probes after methylation-related enzyme digestion, as described herein. In a further embodiment, control probes may be employed to enable a ratio-based comparison against the methylation probe signals generated by the sample DNA, having probe sequences complementary to regions lacking methylation sites or, alternatively, such controls may be run in parallel on known samples and the digestion step omitted, as detailed in the examples herein.

In another embodiment, particularly suited for gene dosage determination as described herein, the sequences of a second set of capture and/or reporter probes are selected so as to be substantially complementary to at least a portion of a known deletion or duplication region (termed a “dosage region”) in a gene or genes of interest. In this manner, the dosage region of interest in a given sample may be assayed for and quantified by comparing the resulting dosage signal against a diploid signal obtained from a known diploid locus in the sample, referred to herein as the “diploid region,” using a second set of probes substantially complementary to the diploid region. Methods and compositions suitable for gene dosage determinations are described more fully in co-pending U.S. patent application Ser. No. 10/093,626, the entire disclosure of which is expressly incorporated by reference herein.

Preferably, the diploid region is selected from a relatively unique region of the genome demonstrating minimal homology with other DNA, thereby minimizing the potential for cross-hybridizing sequence affecting signal strength. Sequence homology is easily ascertained through screening of the human genome through the sequence database maintained by the National Center for Biotechnology Information. As one of skill in the art is well aware, sequence from the non-pseudoautosomal X and Y chromosomal regions should be excluded as dosage varies with gender. Additionally, evidence for potential cell toxicity from over- or under-representation of gene dosage can also be inferred by an examination of chromosomal aberrations in cancer cells (Mitelman Database of Chromosome Aberrations in Cancer (2001). Mitelman F, Johansson B and Mertens F (Eds.), http://cgap.nci.nih.gov/Chromosomes/Mitelman). That is, cancer cells, having lost the normal controls over proliferation and DNA repair and being thus subject to the accumulation of mitotic errors, can indicate specific loci that are more likely to be cell-lethal when present in abnormal copy number. The scarcity of either deletions or duplications of a specific locus in tumor specimens can therefore be taken as evidence that the locus is toxic to cells in abnormal dose and, therefore, will be reliably present in diploid copy number in the vast majority of human cells.

Selection of a diploid region in this manner is particularly suited to the development of assays for somatic dosage abnormalities in mixed-cell populations such as human tissues. Alternatively, so-called “housekeeping genes” can be selected as diploid controls. One of skill in the art will recognize these genes as ones that have been identified as requisite for normal cell growth due to the provision by their product of an essential cell function. Because these genes are also unlikely to be present in other than diploid copy number, they also represent good candidates for diploid loci.

A number of different capture and reporter probes, as described in the examples below, can be included in the same probe mixture. For example, the probe mixture may include two or more probes directed to the same dosage region of interest but having distinct probe complementary sequences. With this embodiment one may guard against the possibility of unknown or rare, undefined SNPs significantly altering the efficacy of hybridization. In a further embodiment, additional probe sets are designed to detect other polymorphisms of interest such as, e.g. one including a known SNP or other polymorphism, with one or more allele-specific detection probes having sequences substantially complementary to the interrogation region upstream and downstream of an interrogation position for which sequence information is desired, but differing in the corresponding interrogation NTPs. In this embodiment, the detection probe sequences are substantially complementary to the sequence surrounding the SNP at the interrogation position, but differ at the corresponding interrogation position with respect to the mutant and wild-type sequences, thereby enabling discrimination between normal and mutant genotypes, as described herein.

The probe complementary sequence that binds to the target will usually be naturally occurring nucleotides, but in some instances the sugar-phosphate chain may be modified, by using unnatural sugars, by substituting oxygens of the phosphate with sulfur, carbon, nitrogen, or the like, by modification of the bases, or absence of a base, or other modification that can provide for synthetic advantages, stability under the conditions of the assay, resistance to enzymatic degradation, etc. In one embodiment, modified nucleotides are incorporated into the probes that do not affect the Tms.

The probes may further comprise one or more labels (including ligand), such as a radiolabel, fluorophore, chemilumiphore, fluorogenic substrate, chemilumigenic substrate, biotin, antigen, enzyme, photocatalyst, redox catalyst, electroactive moiety, a member of a specific binding pair, or the like, that allows for capture or detection of the crosslinked probe. The label may be bonded to any convenient nucleotide in the probe chain, where it does not interfere with the hybridization between the probe and the target sequence. Labels will generally be small, usually from about 100 to 1,000 Da. The labels may be any detectable entity, where the label may be able to be detected directly, or by binding to a receptor, which in turn is labeled with a molecule that is readily detectable. Molecules that provide for detection in electrophoresis include radiolabels, e.g., ³²P, ³⁵S, etc. fluorescers, such as rhodamine, fluorescein, etc., ligand for receptors and antibodies, such as biotin for streptavidin, digoxigenin for anti-digoxigenin, etc., chemiluminescers, and the like. Alternatively, the label may be capable of providing a covalent attachment to a solid support such as bead, plate, slide, or column of glass, ceramic or plastic.

Preferred labels in the present invention include spectral labels such as fluorescent dyes (e.g., fluorescein isothiocyanate, Texas red, rhodamine, dixogenin, biotin, and the like), radiolabels (e.g., ³H, ¹²⁵I, ³⁵S, ¹⁴C, ³²P, ³³P, etc.), enzymes (e.g., horse-radish peroxidase, alkaline phosphatase, etc.), spectral calorimetric labels such as colloidal gold or colored glass or plastic (e.g. polystyrene, polypropylene, latex, etc.) beads. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. Thus, a wide variety of labels may be used, with the choice of label depending on sensitivity required, ease of conjugation with the compound, stability requirements, available instrumentation, and disposal provisions.

The label may be coupled directly or indirectly to the molecule to be detected according to methods well known in the art. Non-radioactive labels are often attached by indirect means. Generally, a ligand molecule (e.g., biotin) is covalently bound to a nucleic acid such as a probe, primer, amplicon, YAC, BAC or the like. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with labeled, anti-ligands. Alternatively, any haptenic or antigenic compound can be used in combination with an antibody. Labels can also be conjugated directly to signal generating compounds, e.g., by conjugation with an enzyme or fluorophore or chromophore.

Means of detecting labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems which are widely available. In general, a detector which monitors a probe-target nucleic acid hybridization is adapted to the particular label which is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising a nucleic acid array with particular set of probes bound to the array is digitized for subsequent computer analysis.

Fluorescent labels are preferred labels, having the advantage of requiring fewer precautions in handling, and being amendable to high-throughput visualization techniques. Preferred labels are typically characterized by one or more of the following: high sensitivity, high stability, low background, low environmental sensitivity and high specificity in labeling. Fluorescent moieties, which are incorporated into the labels of the invention, are generally known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes and flavin. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl I-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2--methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. Many fluorescent tags are commercially available from SIGMA chemical company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

In an alternative embodiment, the probes may further comprise one or more crosslinking compounds. There are extensive methodologies for providing crosslinking upon hybridization between the probe and the target to form a covalent bond. Conditions for activation may include photonic, thermal, and chemical, although photonic is the primary method, but may be used in combination with the other methods of activation. Therefore, photonic activation will be primarily discussed as the method of choice, but for completeness, alternative methods will be briefly mentioned.

The probes will have from 1 to 5 crosslinking agents, more usually from about 1 to 3 crosslinking agents. The crosslinking agents must be capable of forming a covalent crosslink between the probe and target sequence, and will be selected so as not to interfere with the hybridization. In a preferred embodiment, the crosslinking agents in the probe will be positioned across from a thymine (T), cytosine (C), or uracil (U) base in the target sequence.

For the most part, the compounds that are employed for crosslinking will be photoactivatable compounds that can form covalent bonds with a base, particularly a pyrimidine. These compounds will include functional moieties, such as coumarin, as present in substituted coumarins, furocoumarin, isocoumarin, bis-coumarin, psoralen, etc.; quinones, pyrones, α,β-unsaturated acids; acid derivatives, e.g., esters; ketones; nitriles; azido compounds, etc. A large number of functionalities are photochemically active and can form a covalent bond with almost any organic moiety. These groups include carbenes, nitrenes, ketenes, free radicals, etc. One can provide for a scavenging molecule in the bulk solution, normally excess non-target nucleic acid, so that probes that are not bound to a target sequence will react with the scavenging molecules to avoid non-specific crosslinking between probes and target sequences. Carbenes can be obtained from diazo compounds, such as diazonium salts, sulfonylhydrazone salts, or diaziranes. Ketenes are available from diazoketones or quinone diazides. Nitrenes are available from aryl azides, acyl azides, and azido compounds. For further information concerning photolytic generation of an unshared pair of electrons, see Schoenberg, Preparative Organic Photochemistry, 1968.

Another class of photoactive reactants are inorganic/organometallic compounds based on any of the d- or f-block transition metals. Photoexcitation induces the loss of a ligand from the metal to provide a vacant site available for substitutions. Suitable ligands include nucleotides. For further information regarding the photosubstitution of these compounds, see Geoffrey and Wrighton, Organometallic Photochemistry, 1979.

In one preferred embodiment, the crosslinking agent comprises a coumarin derivative as described in co-pending U.S. patent application Ser. No. 09/390,124 and in U.S. Pat. No. 6,005,093, the disclosures of which are incorporated herein in their entirety. Briefly, with this embodiment the probes of the present invention benefit from having one or more photoactive coumarin derivatives attached to a stable, flexible, (poly)hydroxy hydrocarbon backbone unit. Suitable coumarin derivatives are derived from molecules having the basic coumarin ring system, such as the following: (1) coumarin and its simple derivatives; (2) psoralen and its derivatives, such as 8-methoxypsoralen or 5-methoxypsoralen (at least 40 other naturally occurring psoralens have been described in the literature and are useful in practicing the present invention); (3) cis-benzodipyrone and its derivatives; (4) trans-benzodipyrone and its derivatives; and (5) compounds containing fused coumarin-cinnoline ring systems. All of these molecules contain the necessary crosslinking group (an activated double bond) to crosslink with a nucleotide in the target strand.

Another preferred embodiment utilizes the aryl-olefin derivatives as the crosslinking agent, as described in U.S. patent application Ser. No. 09/189,294 and corresponding U.S. Pat. No. 6,303,799, the disclosures of which are incorporated herein in their entirety. In this embodiment, the double bond of the aryl-olefin unit is a photoactivatable group that covalently crosslinks to suitable reactants in the complementary strand. Thus, the aryl-olefin unit serves as a crosslinking moiety and is attached via a linker to a suitable backbone moiety incorporated into the probe sequence.

The probes may be prepared by any convenient method, most conveniently synthetic procedures, where the crosslinker-modified nucleotide is introduced at the appropriate position stepwise during the synthesis. Alternatively, the crosslinking molecules may be introduced onto the probe through photochemical or chemical monoaddition. The above patent disclosures provide specific teachings regarding the incorporation of coumarin and aryl-olefin derivatives, which are incorporated by reference herein. Linking of various molecules to nucleotides is well known in the literature and does not require description here. See, for example, Oligonucleotides and Analogues: A Practical Approach, Echstein (Ed.), 1991.

The probe and target will be brought together in an appropriate medium and under conditions that provide for the desired stringency to provide an assay medium. Therefore, usually buffered solutions will be employed, employing chemicals, such as citrate, sodium chloride, Tris, EDTA, EGTA, magnesium chloride, etc. See, for example, Sambrook et al., Molecular Cloning: A Laboratory Manual, 1988, for a list of various buffers and conditions, which is not an exhaustive list. Solvents may be water, formamide, DMF, DMSO, HMP, alkanols, and the like, individually or in combination, usually aqueous solvents. Temperatures may range from ambient to elevated temperatures, usually not exceeding about 100° C., more usually not exceeding about 90° C. Usually, the temperature for photochemical and chemical crosslinking will be in the range of about 20 to 70° C. For thermal crosslinking, the temperature will usually be in the range of about 70 to 120° C.

The amount of target nucleic acid in the assay medium will generally range from about 0.1 yoctomole to about 100 picomoles, more usually 1 yoctomole to 10 picomoles. The concentration of sample nucleic acid will vary widely depending on the nature of the sample. Concentrations of sample nucleic acid may vary from about 0.01 femtomolar to 1 micromolar. Similarly, the ratio of probe to target nucleic acid in the assay medium may vary, or be varied widely, depending upon the amount of target in the sample, the number and types of probes included in the probe mixture, the nature of the crosslinking agent, the detection methodology, the length of the complementarity region(s) between the probe(s) and the target, the differences in the nucleotides between the target and the probe(s), the proportion of the target nucleic acid to total nucleic acid, the desired amount of signal amplification, the incorporation of crosslinking agents, or the like. The probe(s) may be about at least equimolar to the target but are usually in substantial excess. Generally, the probe(s) will be in at least 10-fold excess, and may be in 10⁶ fold excess, usually not more than about 10¹²-fold excess, more usually not more than about 10⁹-fold excess in relation to the target. The ratio of capture probe(s) to reporter probe(s) in the probe mixture may also vary based on the same considerations.

Conveniently the stringency will employ a buffer composed of about 1× to 10×SSC or its equivalent. The solution may also contain a small amount of an innocuous protein, e.g., serum albumin, β-globulin, etc., generally added to a concentration in the range of about 0.5 to 2.5%. DNA hybridization may occur at elevated temperature, generally ranging from about 20 to 70° C., more usually from about 25 to 60° C. The incubation time may be varied widely, depending upon the nature of the sample, generally being at least about 5 minutes and not more than 6 hours, more usually at least about 10 minutes and not more than 2 hours.

In the crosslinking embodiment, after sufficient time for hybridization to occur, the crosslinking agent may be activated to provide crosslinking. As noted previously above, the activation may involve illumination, heat, chemical reagent, or the like, and will occur through actuation of an activator, e.g., a means for introducing a chemical agent into the medium, a means for modulating the temperature of the medium, a means for irradiating the medium, and the like. If the activatable group is a photoactivatable group, the activator will be an irradiation means where the particular wavelength that is employed may vary from about 250 to 650 nm, more usually from about 300 to 450 nm. The illumination power will depend upon the particular reaction and may vary in the range of about 0.5 to 250 W. Activation may then be initiated immediately, or after a short incubation period, usually less than 1 hour, more usually less than 0.5 hour. With photoactivation, usually extended periods of time will be involved with the activation, where incubation is also concurrent. The photoactivation time will usually be at least about 1 minute and not more than about 2 hours, more usually at least about 5 minutes and not more than about 1 hour.

The purpose of introducing the covalent crosslink between the probes and target DNA is to raise effectively the Tm of the complex above that attained by hydrogen bonding alone. This property allows wash steps to be performed at greater stringency than under initial hybridization conditions, thereby markedly reducing non-specific binding. Thus, the methods of the present invention provide hybridization complexes in which the probe(s) and target sequence(s) are covalently linked to one another, not just hydrogen bonded together. Therefore, harsher conditions that will disrupt any undesirable, nonspecific background binding, but will not break the covalent bond(s) linking the probe to its target sequence, may be employed. For example, washes with urea solutions or alkaline solutions could be used. Heat could also be used. Accordingly, with this embodiment the covalent linkage provides for a significant improvement in the signal-to-noise ratio of the assay.

As described above, high-stringency conditions for the washing step generally employ low ionic strength and high temperature, or alternatively a denaturing agent, such as formamide. In a preferred embodiment, the wash conditions are 1×SSC/0.1% Tween 20 at room temperature (20-25° C.). In another preferred embodiment, the wash conditions are 50% formamide/0.5% Tween 20/0.1×SSC at room temperature (20-25° C.).

After crosslinking of the hybridized probes in the probe mixture, if such crosslinking agents are present, the label(s) incorporated into the probe(s) may be detected. As noted above, a number of different labels that can be used with the probes are known in the art. In the preferred embodiment, one or more capture probes having as a label a member of a specific binding pair, e.g., biotin, are combined with one or more reporter probes having a label that provides a detectable signal. In a preferred embodiment described herein, the reporter probe is polyfluoresceinated to provide for increased signal generation. One may also use a substrate such as AttoPhos, as described herein, or other substrates that produce fluorescent products. With the present invention, the same sample can be contacted with different probe mixtures in different wells of the same microtiter plate in order to assay concurrently for methylation status as well as gene dosage abnormalities such as deletions and duplications, and sequence differences such as SNPs.

In an alternative embodiment, the capture probes described herein may be linked covalently to a solid support prior to performance of the assay. In one such embodiment, a micro-formatted multiplex or matrix device may be used (e.g., DNA chips) (Barinaga, Science 1991; 253:1489; Bains, Bio/Technology 1992; 10:757-8). These methods usually attach specific DNA sequences to very small specific areas of a solid support, such as micro-wells of a DNA chip. In one variant, the methylation assay of the present invention is adapted to solid phase arrays for the rapid and specific detection of multiple methylation sites. A plurality of capture probes directed to a plurality of methylation sites of interest can be linked to a solid support and hybridized with a sample and corresponding sets of reporter probes. The sample will have been previously digested with one or more methylation-sensitive enzymes, and thus the hybridization and subsequent detection of the corresponding reporter probes will be indicative of the methylation status at each site included in the array.

Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPS TM” arrays) can include millions of defined probe regions on a substrate having an area of about 1 cm² to several cm², thereby incorporating sets of from a few to millions of probes.

The construction and use of solid phase nucleic acid arrays to detect target nucleic acids is well described in the literature. See, Fodor et al., Science 1991; 251:767-777; Sheldon et al., Clin. Chem. 1993; 39(4):718-9; Kozal et al., Nat. Med. 1996; 2(7): 753-9; and Hubbell U.S. Pat. No. 5,571,639. See also, Pinkel et al. PCT/US95/16155 (WO 96/17958). In brief, a combinatorial strategy allows for the synthesis of arrays containing a large number of probes using a minimal number of synthetic steps. For instance, it is possible to synthesize and attach all possible DNA 8 mer oligonucleotides (65,536 possible combinations) using only 32 chemical synthetic steps. In general, VLSIPS TM procedures provide a method of producing 4ⁿ different oligonucleotide probes on an array using only 4n synthetic steps.

Light-directed combinatorial synthesis of oligonucleotide arrays on a glass surface is performed with automated phosphoramidite chemistry and chip masking techniques similar to photoresist technologies in the computer chip industry. Typically, a glass surface is derivatized with a saline reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

A 96-well automated multiplex oligonucleotide synthesizer (A.M.O.S.) has also been developed and is capable of making thousands of oligonucleotides (Lashkari et al., PNAS 1995; 93:7912). Existing light-directed synthesis technology can generate high-density arrays containing over 65,000 oligonucleotides (Lipshutz et al., BioTech. 1995; 19:442.

Combinatorial synthesis of probe sequences at different locations on the array is determined by the pattern of illumination during synthesis and the order of addition of coupling reagents. Monitoring of hybridization of reporter probes to the array is typically performed with fluorescence microscopes or laser scanning microscopes. In addition to being able to design, build and use probe arrays using available techniques, one of skill is also able to order custom-made arrays and array-reading devices from manufacturers specializing in array manufacture. For example, Affymetrix Corp., in Santa Clara, Calif. manufactures DNA VLSIP TM arrays.

DNA methylation status as well as a diverse range of polymorphisms in one or more target sequences can be determined in parallel in accordance with the subject protocols. Clinical diagnostics is improved substantially with the present invention by the ability to assay methylation status simultaneously with other mutational mechanisms of human genetic variation in a single platform, including both gene dosage and sequence abnormalities. The resulting genetic profile obtained for a given locus will be more complete and can be used for risk profiling, chemopredictive testing, disease profiling, and pharmacogenetic testing, as well as for determining genetic mutations, genetic diseases, genotyping for trait analysis, and genotyping of other polymorphic sequences in humans, plants, and animals.

Specific target sequences of interest include the 15q11-q13 chromosomal region. Parental-origin-specific DNA methylation is observed in the 15q11-q13 chromosomal region (Prader-Willi syndrome (PWS)/Angelman Syndrome (AS) region) (M. Velinov, et al., Mol. Genet. And Metab. 2000; 69:81-83). The DNA methylation patterns are abnormal in both PWS and AS; therefore methylation tests can be used to identify all PWS cases and about 75% of AS cases (M. Velinov, et al., Mol. Genet. And Metab. 2000; 69:81-83).

In adult human tissues, a HpaII and CfoI restriction site at the PW71 (D15S63) locus in the PWS region in chromosome 15 are methylated on the maternal chromosome, but unmethylated on the paternal chromosome (B. Dittrich et al., Hum. Molec. Genet. Vol.2 1993; 12:1995-1999). The HpaII site is part of a sequence with high homology to the long terminal repeat of human endogenous retroviruses. Based on this methylation imprint, one diagnostic test for PWS is a Southern blot hybridization of HindIII and HpaII digested DNA. Normal individuals reveal a 6.6 kb fragment that is derived from the maternal chromosome and a 4.7 kb fragment that is derived from the paternal chromosome (B. Dittrich et al., Hum. Molec. Genet. Vol.2, 1993; 12:1995-1999). Patients with PWS typically lack the 4.7 kb fragment (B. Dittrich et al., Hum. Genet. 1992; 313-315).

Human genomic loci known to be subject to germline dosage and methylation patterns associated with abnormal phenotypes include the following:

TABLE 2

| Chromo- | | | Assay Locus |
|---|---|---|---|
| somal | | Mechanism of | for Dosage |
| Locus | Phenotype | Mutation | and Methylation |
| 15q11-13 | Prader- | Paternal deletions and | Maternal methyl- |
| | Willi/Angelman | maternal UPD (PWS); | ation of SNRPn |
| | syndromes | maternal deletions and | exon 1 |
| | | paternal UPD (AS) | |
| 11p14 | Beckwith- | Paternal duplications, | Paternal methyl- |
| | Wiedemann | paternal UPD, “loss- | ation of H19 |
| | syndrome | of-imprinting muta- | promoter |
| | | tions” maternal H19 | |
| 6q24 | Transient | Paternal duplications, | Maternal methyl- |
| | Neonatal | paternal UPD | ation of CpG |
| | Diabetes Mellitus | | island in |
| | | | HYMA1/ZAC |

In human cancers, loss of expression of tumor suppressor genes is regularly associated with cancer progression. Deletions, loss of entire chromosomes and methylation of CpG islands leading to repression of transcription are all common somatic mutations found in tumor tissues. Genes for which both gene loss and abnormal methylation patterns are observed in cancer cells include the following:

TABLE 3

| Gene | Cancer Type |
|---|---|
| hMLH1 | Colorectal, gastric |
| P14ARF and p16INK4a | Colorectal, melanoma, ovarian, lung, |
| | glioblastoma |
| CDKN2b | Hematologic malignancy |
| VHL | Renal |
| RB1 | Retinoblastoma |
| p53 | Lung |
| E-cadherin | Esophageal |
| GSTP1 | Prostate |
| RARbeta2 | Prostate |
| FHIT | Lung, breast |
| p73 | Acute lymphoblastic leukemia |

Reviews: Jones P A, Laird P W, Nat. Genet. 1999; 21:163-167; Hall J G, Annu. Rev. Med. 1997; 48:35-44.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXPERIMENTAL

### Example 1

### Combined Gene Dosage and Methylation Assay Using Crosslinking Technology

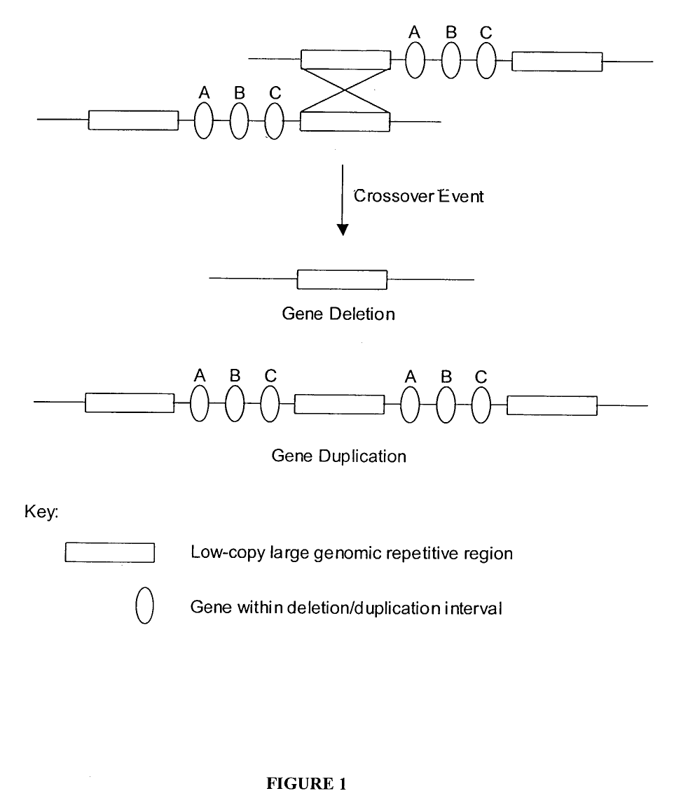

The deletion/duplication locus at 15q11-13 serves as a model system for development of techniques for concurrent assessment of gene dosage and CpG methylation (reviewed in Hanel and Wevrick, Clin. Genet. 2000; 59:156-64 and Cassidy et al., Am. J. Med. Genet. 2000; 97:136-46). In the first place, the region prone to deletion/duplication is also bounded by low-copy large genomic repetitive regions predisposing to misalignment in meiosis and the subsequent formation of unbalanced crossover events, as illustrated in FIG. 1. The deletion or duplication is typically 3 to 5 mb, depending on which repeats align. In the second place, the entire region is also subject to gametic imprinting. That is, gene expression is controlled by epigenetic modification distinguishing the maternally and paternally derived chromosomes. Certain genes within the critical interval are expressed exclusively from only one chromosome. Gene dosage effects are determined not by the absolute copy number, but by the copy number of expressed genes, i.e., the copy number of genes present on the actively transcribed chromosome. Therefore, the phenotypic effect varies widely depending on the parental origin of the chromosome that is abnormal.

The Prader-Willi and Angelman syndromes are quite different; the former comprises moderate to severe mental retardation, profound obesity and dysmorphic features, while manifestations of the latter include normal growth parameters, severe mental retardation with autistic features and seizure disorder. Patients with both syndromes, however, have the identical deletion of chromosome 15q11-13, but the former phenotype occurs in the setting of a deletion of the paternally derived chromosome, while the latter is associated with the deletion occurring on the chromosome from the mother. The identical phenotypes are produced in the setting of chromosome 15 uniparental disomy, the situation which exists when an offspring has a normal diploid copy number, but both chromosome 15 homologues came from one parent and there is no contribution from the other. In the case of Prader-Willi syndrome, about 70% of cases are accounted for by deletions of the paternal copy of chromosome 15, removing genes transcribed exclusively from the paternal chromosome. The majority of the balance of cases results from maternal disomy for chromosome 15 in which both copies are transcriptionally silent for the paternally expressed genes. Angelman syndrome results from the absence of maternally derived transcripts, either from a maternal chromosome deletion or, more rarely, paternal disomy. The duplication events produce a subtler phenotype, including a form of autism associated with duplications occurring on the maternal chromosome only. This suggests that a gene or genes within the interval as yet to be identified, normally expressed from the maternal chromosome confers the phenotype when present in excess active copy number (Cook et al., Am. J. Hum. Genet. 1997; 60:928-34).

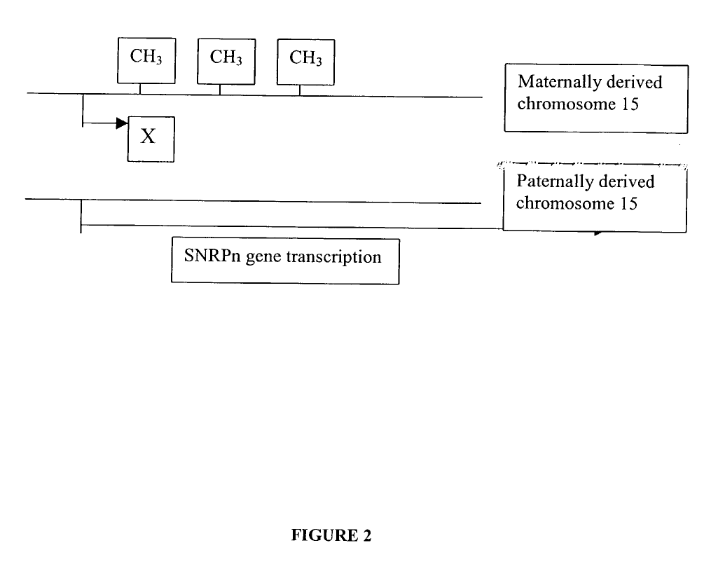

Transcription versus silencing of imprinted genes is associated with characteristic patterns of methylated CpG sites within the region. The SNRPn gene, expressed only from the paternal chromosome and a candidate for at least some of the phenotypic findings of the Prader-Willi syndrome, is preferentially methylated at specific sites of the promoter region and within exon I on the maternal, or inactive, chromosome. In fact, a small number of cases of the Prader-Willi syndrome have been determined to be caused by small deletions including exon I on the paternal chromosome that is associated with conferring a maternal methylation pattern over the rest of the area. These so-called “imprinting center” mutations have the identical effect of altering gene transcription as a deletion of the entire region or uniparental disomy, as illustrated in FIG. 2. For efficient and accurate molecular diagnosis of the Prader-Willi/Angelman and duplication 15q11-13 syndromes, there is a need for a rapid, cost-effective technology that will allow for the parallel ascertainment of gene dosage and methylation status.

A gene-dosage assay for the 15q11-13 region was developed to determine cytosine methylation status and gene copy number within the Prader-Willi/Angelman and duplication 15q11-13 syndrome critical region. A 1980 bp unique genomic sequence from within the duplication/deletion interval including the SNRPn gene exon 1, known to be reliably differentially methylated between the maternally and paternally derived chromosomes 15, has been identified (Zeschnigk M. et al., Hum. Molec. Genet. 1997; 6:387-395). Two separate assays have been designed from within the 15q11-13 region; one allows for ascertainment of overall region copy number (when performed in parallel with an extradeletion control assay) while the other determines the number of copies specifically containing methylated cytosines at the given sites. In accordance with the methods of the present invention, the probes were designed in such a way as to separate the capture sites used in the methylation-sensitive assay from the reporter sequences by methylation-senstive restriction enzyme sites. That is, the reporter probe set comprises sequence 3′ of the methylation-sensitive capture probe sets. The reporter probes were also polyfluoresceinated and, therefore, only four to six are required for ample signal. The two capture probes were designed from sequences separated from the reporter set by two and three HpaII sites, respectively. A second set of capture probes was developed 3′ of the reporter probe set not predicted to be affected by HpaII digestion that are used to determine overall gene copy number.

A control locus was developed from the ANK2 gene locus at 4q25, which served as the diploid control for this assay. The 4q25 and 3′SNRPn assays each contain 4 reporter probes. The 5′SNRPn probe set utilizes the same four reporter probes as does the 3′SNRPn assay, as well as two further reporter probes unique to the 5′SNRPn assay. All reporter probes were polyfluoresceinated with roughly 20-30 molecules of fluorescein per oligonucleotide.

TABLE 4

| Oligonucleotide identification | Sequence 5′-3′ |
|---|---|
| 5′SNRPn-cap A | AXAGCTGACCTTGCCCGCTCCATCGCGTCACTGACCGCTCC |
| | TCAXA |
| 5′SNRPn-cap B | AXATTCCGTTTATTCAGTACTCCAAGTCCTAXA |
| 3′SNRPn-cap A | AXAAATATGAACTTAGACCCCCACCTAAXA |
| 3′SNRPn-cap B | AXAGCCTTTCTTTGCCTATTAGAATTGGATACATTAXA |
| 3′SNRPn/5′SNRPN-rep 1 | FAXATTTTTGCACACACCACTGGCCAXAF |
| 3′SNRPn/5′SNRPN-rep 2 | FAXATGCGCCATAACCACAXTF |
| 3′SNRPn/5′SNRPN-rep 3 | FAXAAGAAAATATCCCTAACTCTAXAF |
| 3′SNRPn/5′SNRPN-rep 4 | FAXATGTCTACCTGTTTTTTAAXAF |
| 5′SNRPn-rep 5 | FAXACCATAAGCAACCTGGGATCAXTF |
| 5′SNRPn-rep 6 | FAXACACTGGCTATTCAATTTTTGTAXAF |
| 4q25-cap A | CXAGGCAAACTCTCTAAATTAATGGTGTTTCCTCTAAXA |
| 4q25-cap B | GGACTTGATTCTAGCAXAAAATGGGGAGCCACCATAXA |
| 4q25-rep 1 | AXAGGGTTATGATTAGTTTAXA |
| 4q25-rep 2 | AXAATACATTGCATCATCTAXA |
| 4q25-rep 3 | AXACTCATAGCCTCTTCCCAGAXA |
| 4q25-rep 4 | AXTGGGTTCTTATATTATGATGTGAXA |

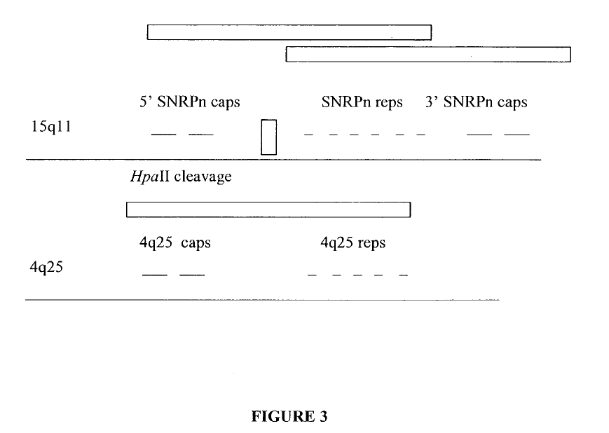

The design of the complete assay was as follows: A single DNA sample was digested with HpaII, precipitated, resuspended in solution, divided into each of 6 wells and probed in duplicate with each of three probe sets: the SNRPn reporters with the cap1 capture probe set (5′SNRPn assay); the SNRPn reporters with the cap2 capture probe set (3′SNRPn assay); and the 4q25 probe set, drawn from sequence lacking HpaII sites. The design of the 3 probe sets is illustrated in FIG. 3.

With unmethylated DNA, the 5′SNRPn reporter probe-target complex is no longer contiguous with the capture-target complex and negligible signal is observed. The 3′SNRPn and 4q25 assays are unaffected by DNA digestion. The 3′ SNRPn/4q25 net sample signal ratio determines the overall 15q11 region copy number. The 5′ SNRPn/3′SNRPn net sample signal ratio determines number of METHYLATED copies of 15.

The 3′SNRPn and 4q25 assays can be performed on lysed leukocyte pellets or extracted DNA for SNRPn locus dosage assessment alone as described below. By performing a HpaII digestion on extracted DNA and assaying with all 3 probe sets, the assay can accurately determine both gene dosage and methylation status simultaneously. Positive and negative controls are created from DNA from a phenotypically normal subject and processed in parallel with the experimental samples with the exception that the HpaII digestion is omitted from each. Controls are assayed in parallel with experimental samples with each probe set, although capture probes are omitted from the negative control sample probe sets. Net sample signals are obtained for experimental and control subjects for each assay by subtracting mean background signal (negative control value) from mean sample and positive control signals. Assessment of 15q11-13 region dosage for experimental samples is performed by obtaining the ratio of 3′SNRPn net sample signals to 4q25 net sample signals (signal ratio, or SR) normalized to that ratio obtained for the positive control sample (normalized signal ratio, or NSR).

Dosage Determination:

$$\begin{matrix}SR = \frac{\{ \begin{matrix}mean of two 3 ’ SNRPn \\ sample signals\end{matrix} \} - \{ \begin{matrix}mean of two 3 ’ SNRPn \\ negative control signals\end{matrix} \}}{\{ \begin{matrix}mean of two 4 q 25 \\ sample signals\end{matrix} \} - \{ \begin{matrix}mean of two 4 q 25 \\ negative control signals\end{matrix} \} } \\ NSR = \frac{SR ( sample )}{SR ( control blood sample )}\end{matrix}$$

The number of methylated SNRPn copies is determined by the ratio of the net 5′SNRPn to net 3′SNRPn signals normalized to that ratio for the positive control sample. It is worth noting that the ratio of 5′SNRPn to 3′SNRPn signals in the control sample will reflect the presence of two apparently methylated copies of SNRPn, or a 1:1 ratio of 5′SNRPn to 3′SNRPn dosage due to the absence of HpaII digestion in the control sample.

Fraction of SNRPn Copies that are Methylated:

$$\begin{matrix}SR = \frac{\{ \begin{matrix}mean of two 5 ’ SNRPn \\ sample signals\end{matrix} \} - \{ \begin{matrix}mean of two 5 ’ SNRPn \\ negative control signals\end{matrix} \}}{\{ \begin{matrix}mean of two 3 ’ SNRPn \\ sample signals\end{matrix} \} - \{ \begin{matrix}mean of two 3 ’ SNRPn \\ negative control signals\end{matrix} \} } \\ NSR = \frac{SR ( sample )}{SR ( control blood sample )}\end{matrix}$$

By performing the above data analysis, a comprehensive profile of the SNRPn locus can be obtained. The following table represents possible profiles that would be expected for particular genotypes:

TABLE 5
Expected results

| | | | PWS | AS | | | |
|---|---|---|---|---|---|---|---|
| | | | dele- | dele- | PWS | | |
| | Normal | | tion | tion | UPD | Trisomy | Trisomy |
| | not | Normal | (pat | (mat | (mat | 2 mat, | 2 pat, |
| | digested | digested | del) | del) | disomy) | 1 pat | 1 mat |
| 5′SNRPn | A | 0.5 A | 0.5 A | 0 | A | A | 0.5 A |
| 3′SNRPn | A | A | 0.5 A | 0.5 A | A | 1.5 A | 1.5 A |
| 4q25 | A | A | A | A | A | A | A |

Experimental Protocol:

Leukocytes were isolated from blood samples using a red cell lysis procedure, as described in Zehnder et al., Clin. Chem. 1997; 43:1703-8. For parallel dosage and methylation assay, genomic DNA was extracted from leukocytes or human lymphoblasts obtained from the Coriell Cell Repository (Puregene). 250-350 ug of DNA was digested overnight with 1 unit/ug DNA of the restriction enzyme HpaII (NEB), precipitated with ethanol, resuspended in leukocyte lysis buffer (0.28 M NaOH) and boiled for 20 minutes to shear the DNA to the desired fragment size. Processed samples were placed into six wells each of a 96 well polypropylene microtiter plate. Each assay plate also contained six negative controls and six positive controls as described above. Three different probe solutions were prepared, each containing the same set of locus specific reporter probes and capture probes as described. All probe mixes were prepared with a final concentration of each capture probe at 0.5 pMole per well and each reporter at 0.2 pMole per well, with the exception of aliquots for the negative controls, from which capture probes were omitted. Aliquots of each probe solution were added in duplicate to each sample well, as well as to negative and positive control wells. Neutralization of the solutions, photo-crosslinking and addition of the strepatavidin coated magnetic beads have been described (ibid). The only significant deviation from the SNP assay procedure involves the high-stringency wash conditions employed for this assay. Following incubation of the crosslinked hybridization mixture with the magnetic beads, the beads were washed first with a pre-wash (0.1% SDS, 0.1×SSC, 0.001% Tween 20), then with the gene dosage high stringency wash (50% formamide, 0.5% Tween 20, 0.1×SSC), and finally with the SNP wash (1×SSC, 0.1% Tween 20). The beads were incubated in the presence of anti-fluorescein antibody-alkaline phosphatase conjugate (DAKO Corp., Carpinteria, Calif.), washed four times and resuspended in Attophos™ (Promega, San Luis Obispo, Calif.) as described (ibid). The fluorescence signal was determined by reading the plate in a microplate fluorometer (Packard Instrument Co., Meriden, Conn.). The data was analyzed as described above.

Experimental Results:

Results are from experiments utilizing DNA from lymphoblastoid cell lines (Coriell Cell Repository) carrying characterized genotypes of the 15q11-13 region:

Data from Assays on 12 PWS, 3 AS, 2 Duplications and 9 Normal Controls

TABLE 6
Dosage Data

| | Geno- | | Ex- | | | | DX | |
|---|---|---|---|---|---|---|---|---|
| DX | type | N | pected | Mean | SD | Range | Range | OOS |
| PWS | pat del | 19 | 0.5 | 0.532 | 0.0843 | 0.36- | 0.35- | 1 |
| | | | | | | 0.69 | 0.65 | |
| AS | mat del | 6 | 0.5 | 0.528 | 0.0402 | 0.46- | 0.35- | 0 |
| | | | | | | 0.58 | 0.65 | |
| | total del | 25 | 0.5 | 0.531 | 0.0753 | 0.36- | 0.35- | 1 |
| | | | | | | 0.69 | 0.65 | |
| NC | normal | 12 | 1 | 0.873 | 0.11 | 0.74- | 0.80- | 4 |
| | | | | | | 1.11 | 1.15 | |

TABLE 7
Methylation Data

| | Geno- | | Ex- | | | | DX | |
|---|---|---|---|---|---|---|---|---|
| DX | type | N | pected | Mean | SD | Range | Range | OOS |
| PWS | pat del | 19 | 1 | 0.883 | 0.167 | 0.68- | >0.75 | 2 |
| | | | | | | 1.33 | | |
| AS | mat del | 6 | 0 | 0.53 | 0.127 | 0.32- | <0.60 | 1 |
| | | | | | | 0.71 | | |
| NC | normal | 12 | 0.5 | 0.483 | 0.128 | 0.28- | 0.35- | 3 |
| | | | | | | 0.67 | 0.65 | |

The only significant deviation from expected values is seen in the case of the maternal deletions, in which a mean value of 0.53 was obtained as compared with an expected value of 0. This deviation most likely reflects the fact that the experimentally determined background signal for each assay is an approximation of the true background; slight differences between experimental and true backgrounds are predicted to affect NSR values closer to zero than nearer to one. Despite the deviation from expected, there is a clear demarcation between values obtained for the deleted samples, affording accurate discrimination between maternal and paternal deletions. The results indicate that the crosslinking technology has been successfully applied to the determination of SNRPn gene dosage and chromosomal parent-of-origin. The methodology represents a substantial improvement over current techniques.

### Example 2

### Combined Gene Dosage and Methylation Assay without Crosslinking

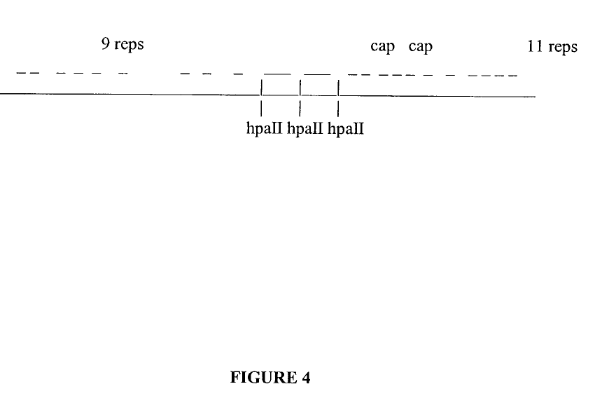

An alternate methodology for determination of methylation and dosage status at 15q11-13 does not require the crosslinking technology. In this embodiment, two capture probes of 44 and 46 base pairs biotinylated at the 3′ end and 20 reporter probes of 20 to 32 base pairs fluoresceinated either at both the 5′ end or one each at the 5′ and 3′ end are employed. The capture probes were designed in such a way as to separate the capture sites from the reporter sequences by the differentially methylated HpaII sites; in this embodiment, the reporter probes are located within 750 kb to either side of the capture probe/HpaII locus, as shown in FIG. 4.

The control locus assay at 4q25 from Example 1 serves as the control for this assay as well. In this embodiment, the assay is performed using the SNRPn probe mixture on separate aliquots of sample material, one of which has been predigested with HpaII and one of which has been treated identically with the exception of omission of the enzyme. A third aliquot, also undigested, is assayed with the 4q25 control locus probe set. Comparison of the signals obtained between both undigested samples allows for assessment of overall gene dosage, as has been described in Example 1. Comparison of signal obtained from the digested and undigested samples assayed with the SNRPn probes will allow the determination of methylation status. The methylation-sensitive enzyme HpaII will only cleave unmethylated restriction sites, thereby removing reporter sequences from the capture probe/genomic DNA complex on unmethylated, but not on methylated, chromosomes 15. Therefore, signal is only obtained from chromosomes posessing methylated cytosine residues. Quantitative analysis of the digested and undigested SNRPn signals accurately identifies methylated locus dosage in parallel with overall gene copy number.

The assay itself is performed with the identical protocol as that developed for the crosslinking assay in Example 1 with the following exceptions. Extracted human DNA is aliquoted equally into three tubes prior to digestion; each tube receives HpaII buffer and either HpaII or water to achieve equal volumes. Samples are incubated overnight at 37 degrees and precipitated and processed as described in Example 1. Two 125 ul aliquots are removed from each tube and placed in wells of a 96 well plate. The HpaII digested sample is assayed with the SNRPn probe mixture, while the undigested samples are assayed with the SNRPn probes and the 4q25 probes separately in each of two wells. Normal human genomic DNA processed in parallel without DNA digestion is assayed with each of the SNRPn and 4q25 probe mixtures as a normalization control, as in Example 1. The assays are performed identically as in Example 1, with the exception of the post-bead addition wash steps, immediately prior to addition of anitfluorescein antibody. A less stringent wash solution is used in place of the 50% formamide wash solution described in Example 1, which allows for preservation of the non-covalent probe/target hybridization complexes. The remainder of the assay is performed identically to that in Example 1.

### Example 3

### Methylation Assay of the p53 Gene for Use in Lung Cancer Screening

Hypomethylation of the transcribed sequence of the tumor suppressor gene p53 has been demonstrated in several different tumor types. In one study, evidence of somatic mosaicism for this epigenetic modification in peripheral blood lymphocytes was associated with a two-fold increased risk for lung cancer in male smokers (Woodson et al., Cancer Epidemiol Biomarkers Prev. 2001; 10(1):69-74). Therefore, a high-throughput method for determining p53 exon 5-8 CpG methylation would be of utility in clinical diagnostics. The method described below represents a substantial improvement over current methodologies.

A 1080 base-pair target sequence was identified from the p53 gene sequence of exon 5 through intron 7 (reverse complement of nucleotides 1621-2700 of Genbank accession number AF136270) containing 4 HpaII-sensitive CpG methylation sites known to be associated with malignant transformation-specific hypomethylation. Six polyfluoresceinated reporter probe sequences and four biotinylated capture probe sequences have been selected, each containing two coumarin-based photocrosslinking moieties. The capture probes are designed to incorporate a minimum of 32 base pairs of sequence each in order to obviate the effects of undefined polymorphisms. Probe sequences are given in the table below. Nucleotide sequences correspond to the GenBank sequence given above. The letter “X” denotes the crosslinking nucleotide.

TABLE 8

| Probe ID | Nucleotide Position | Probe Sequence (5′ to 3′) |
|---|---|---|
| CAP1 | 1695-1664 | AXCCTCCGTCATGTGCTGTGACTGCTTGTAXA |
| CAP2 | 1936-1894 | AXACCTCAGGCGGCTCATAGGGCACCACCACACTATGTCGAXA |
| CAP3 | 2661-2623 | AXAGGCTGGGGCACAGCAGGCCAGTGTGCAGGGTGGCXA |
| CAP4 | 2699-2664 | AXATCGGTAAGAGGTGGACCCAGGGGTCAGAGGCXA |
| REP1 | 1796-1774 | AXAGGCCTGGGGACCCTGGGCXA |
| REP2 | 1816-1799 | AXAGCAATCAGTGAGGXA |
| REP3 | 1842-1821 | AXGATGCTGAGGAGGGGCCAXA |
| REP4 | 1877-1860 | AXATACTCCACACGCAXA |
| REP5 | 1956-1939 | AXAGACCCCAGTTGCAXA |
| REP6 | 1993-1968 | AXAGGGCCACTGACAACCACCCTTXA |

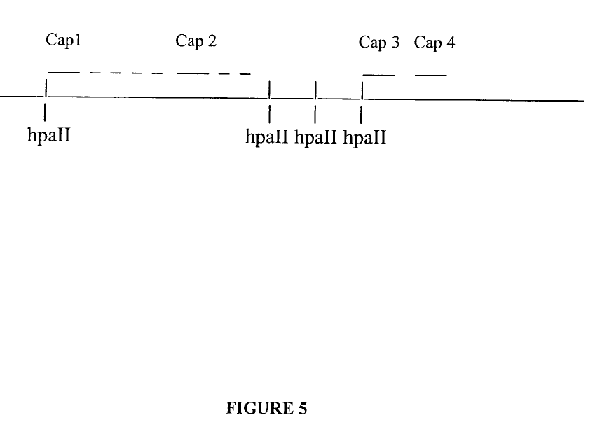

As shown in FIG. 5, the reporter sequences and the methylation-insensitive capture probes (CAP1 and CAP2), are separated from the methylation-sensitive capture sequences (CAP3 and CAP4), by three HpaII sites. Reporter probe sequences are indicated by short lines.

The assay is performed using the identical protocol given for the 15q11-13 methylation assay (see Example 1). Genomic DNA is extracted from peripheral blood leukocytes, digested with the restriction endonuclease HpaII, and then precipitated. The DNA is resuspended in an alkaline solution and denatured by heating. The DNA is then aliquotted into each of 4 wells of a 96-well plate. Two probe sets are created, each containing the complement of 6 polyfluoresceinated reporter probes. Whereas one probe set contains the methylation-insensitive capture probe set (CAP1 and CAP2), the other probe set contains the methylation-sensitive capture probe set (CAP3 and CAP4). The probe sets are added to hybridization mixtures, whose components have been described in Example 1. 50 μL aliquots of either the methylation-insensitive or -sensitive probe mixture are added to the DNA in duplicate wells. Hybridization, photocrosslinking, signal amplification/detection are performed as described in Example 1.

Negative and positive control samples are run in parallel with each assay in order to assess background (denaturation solution only) and relative probe signal strength (undigested DNA sample), respectively. Relative p53 methylation is determined from the ratio of the background-corrected methylation-sensitive probe set signal to the background-corrected methylation-insensitive probe set signal, normalized to that ratio obtained using undigested DNA as a control, as described in Example 1. The data can be compared against that obtained by Woodson et al. as follows: The normalized net signal ratio of methylation-sensitive to methylarion-insensitive signal for samples is expected to be close to 1.0, consistent with complete methylation of the p53 gene in exons 5-8. In their study, a value of less than 0.75 is interpreted as hypomethylation of p53 exons 5-8 and conferred a potential 2-fold risk of developing lung cancer in male smokers.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the following claims.

What is claimed is:

1. A method for determining the methylation status of a target nucleic acid sequence in a sample, wherein said target nucleic acid sequence comprises a first and a second binding domain and at least one methylation site, said method comprising the steps of:
a) adding a methylation-related digestion enzyme to said sample;
b) adding a capture probe having a sequence substantially complementary to at least a portion of said first binding domain and a reporter probe having a sequence substantially complementary to at least a portion of said second binding domain, wherein said first and second binding domains are separated by said methylation site in said target sequence;
c) capturing said capture probe; and
d) detecting said reporter probe to determine methylation status at said methylation site.

2. The method of claim 1, wherein said methylation-related enzyme is a methylation-sensitive enzyme, and said detection of said reporter probe indicates methylation at said methylation site.

3. The method of claim 1, wherein said methylation-related enzyme is a methylation-dependent enzyme, and said detection of said reporter probe indicates a lack of methylation at said methylation site.

4. The method of claim 1, wherein said capture and reporter probes comprise first and second detectable labels respectively.

5. The method of claim 2, wherein said first detectable label is a capture molecule.

6. The method of claim 2, wherein said second detectable label is a reporter molecule.

7. The method of claim 1, wherein said capture and reporter probes are crosslinkable probes comprising at least one crosslinking agent.

8. The method of claim 7, wherein said crosslinkable probes are activated to crosslink to their respective binding domains prior to capture of said capture probe.

9. The method of claim 8, wherein said crosslinkable probes comprise a photo-activatible crosslinking agent.

10. A method for genotyping a target sequence in a sample, wherein said target sequence comprises a dosage region and a methylation site flanked by first and second binding domains, said method comprising:
a) adding a methylation-related digestion enzyme to said sample;
b) hybridizing said first and second binding domains to a first probe mixture to form at least one first hybridization complex, said first probe mixture comprising at least one methylation capture probe having a sequence substantially complementary to at least a portion of said first binding domain and at least one methylation reporter probe having a sequence substantially complementary to at least a portion of said second binding domain, wherein said first and second binding domains are separated by said methylation site in said target sequence;
c) hybridizing said dosage region to a second probe mixture to form at least one second hybridization complex, said second probe mixture comprising at least one dosage reporter probe comprising a detectable label capable of producing a dosage signal and a sequence substantially complementary to at least a portion of said dosage region;
d) capturing said at least one methylation capture probe, and
e) determining the copy number of said dosage region based on the ratio of said dosage region to a diploid signal and detecting said methylation reporter probe to determine the methylation status of the target.

11. The method of claim 10, comprising the additional steps of hybridizing a third probe mixture to a diploid region in said sample and performing said detecting step to obtain said diploid signal; wherein said third probe mixture comprises at least one diploid reporter probe having a sequence complementary to at least a portion of said diploid region and a detectable label capable of producing said diploid signal.

12. The method of claim 10, wherein said methylation-related enzyme is a methylation-sensitive enzyme, and said detection of said reporter probe indicates methylation at said methylation site.

13. The method of claim 10, wherein said methylation-related enzyme is a methylation-dependent enzyme, and said detection of said reporter probe indicates a lack of methylation at said methylation site.

14. The method of claim 10, wherein said capture and reporter probes are crosslinkable probes comprising at least one crosslinking agent.

15. The method of claim 14, wherein said crosslinkable probes are activated to crosslink to their respective binding domains prior to capture of said capture probe, whereby said first hybridization complex becomes covalently crosslinked when said first and second binding domains are present in said sample, and said second hybridization complex becomes covalently crosslinked when said dosage region is present in said sample.

16. The method of claim 15, wherein said crosslinkable probes comprise a photo-activatible crosslinking agent.

17. A method for genotyping a target sequence in a sample, wherein said target sequence comprises a methylation-site flanked by first and second binding domains and an interrogation region comprising an interrogation position, said method comprising:
a) adding a methylation-related digestion enzyme to said sample;
b) hybridizing said first and second binding domains to a first crosslinkable probe mixture to form at least one first hybridization complex, said first crosslinkable probe mixture comprising at least one methylation capture probe having a sequence substantially complementary to at least a portion of said first binding domain and a methylation reporter probe having a sequence substantially complementary to at least a portion of said second binding domain, wherein said first and second binding domains are separated by said methylation site in said target sequence;
c) hybridizing said interrogation region to a second crosslinkable probe mixture to form at least one second hybridization complex, said second crosslinkable probe mixture comprising at least one allele-specific detection probe comprising a crosslinking agent, a detectable label capable of producing an interrogation signal and a sequence substantially complementary to the sequence upstream and downstream of the interrogation position in said interrogation region;
d) activating said crosslinking agent, whereby said first hybridization complex becomes covalently crosslinked when said first and second binding domains are present in said sample, and said second hybridization complex becomes covalently crosslinked when said detection position is perfectly complementary to said interrogation position;
e) washing said crosslinked first and second hybridization complexes at least once under high-stringency conditions; and
f) detecting said at least one methylation reporter probe to determine the methylation status of the target and detecting said interrogation signal to determine the identity of said interrogation position.
